(19) United States
(12) Patent Application Publication
(10) Pub. No.: US 2004/0235056 A1
(43) Pub. Date: Nov. 25, 2004

(54) Method of assembling particle libraries

(76) Inventors: Thomas E. Mallouk, State College, PA (US); Ramnarayanan Ramanathan, State College, PA (US); Richard R. Willis, Cary, IL (US); Ralph D. Gillespie, Gurnee, IL (US)

Correspondence Address: JOHN G TOLOMEI, PATENT DEPARTMENT, UOP LLC, 25 EAST ALGONQUIN ROAD, P O BOX 5017, DES PLAINES, IL, 60017-5017, US

(21) Appl. No.: 10/834,479

(22) Filed: Apr. 29, 2004

Related U.S. Application Data
(60) Provisional application No. 60/468,500, filed on May 7, 2003.

(51) Int. Cl. G01N 33/53; G03G 15/09; G01N 33/543

(52) U.S. Cl. 435/7.1; 436/518

(57) ABSTRACT

A method for assembling arrays of materials where each array contains different materials on or within a particle and the location of each particle at a spatially identifiable location in the array determines the material associated with that particle, has been developed. Assembling of the arrays begins with the delivery, of a particle and a component to a first isolated location in first region. A second isolated location on the first region also receives another particle and component to form at least two particle-component pairs in the first region. At least one of the particle-component pairs is selectively transferred to an isolated location in a second region where the particle-component pair receive an additional component thereby producing a first material associated with the first particle-component pair and a second material associated with the second particle-component pair.

### CROSS-REFERENCE TO RELATED APPLICATION

This application claims priority from Provisional Application Serial No. 60/468,500 filed May 7, 2003, the contents of which are hereby incorporated by reference.

### STATEMENT REGARDING FEDERALLY SPONSORED RESEARCH OR DEVELOPMENT

This invention was made under the support of the United States Government, Department of Commerce, National Institute of Standards and Technology (NIST), Advanced Technology Program, Cooperative Agreement Number 70NANB9H3035. The United States Government has certain rights in the invention.

### FIELD OF THE INVENTION

The invention is a method for assembling particle libraries wherein the particles having known components are in known locations of a region. The method incorporates transferring at least a portion of the particles from a first region to a second region.

### BACKGROUND OF THE INVENTION

In classic split-pool procedures, each particle in a pool of particles can be uniformly coated with the reactant component(s) of interest and, thereafter, reacted. This is readily done, for example, by using a series of vessels each of which contains a solution of a particular reactant component. The particles are equally divided into groups corresponding to the number of components used to generate the array of materials. Each group of particles is then added to one of the vessels wherein a coating of one of the components in solution forms on the surface of each particle. The particles are then pooled together into one group and heated to produce a dry component layer on the surface of each of the particles. The process is repeated several times to generate an array of different reaction components on each of the particles. Once the components of interest have been deposited on the particles, the particles are reacted to form an array of materials. All of the particles may or may not be reacted under the same reaction conditions. To determine the history of the components deposited on a particular particle, spectroscopic or other analytical techniques can be used. Alternatively, each particle can have a tag which indicates the history of components deposited thereon as well as their stoichiometries. The tag can be, for example, a binary tag etched into the surface of the particle so that it can be read using spectroscopic techniques. Each of the individual particles or pellets can be screened for materials having useful properties.

Two drawbacks of the classic split and pool techniques are that either analysis of a formed material or tagging of formed materials is required, and the materials come into direct contact with one another during assembly. Analyzing the materials after formation or tagging materials require additional steps and add a level of complexity that is not necessary in the present invention. Furthermore, in the classic split-pool technique, the individual materials come in direct contact with one another, which may lead to cross contamination problems. Minimizing cross contamination may require additional steps in the assembly of the materials such as heating before pooling.

The invention eliminates the need for analyzing the materials made in order to learn the components added. Some analysis may still be required such as to determine whether a novel structure has been developed, but the components delivered to spatially identifiable locations on the region are known. Also, by isolating particle-components from other particle-components retained by the region, cross contamination between particle-components is minimized.

### BRIEF DESCRIPTION OF THE INVENTION

This invention is a method for assembling one or more arrays of materials where each array contains different materials on or within a particle and the location of each particle at a spatially identifiable location in the array determines the material associated with that particle. Assembling of the arrays begins with the delivery, in any order, of a particle and a component of the material formed on or within the particle to a first isolated location in first region. A second isolated location on the first region also receives another particle and component of the material to form at least two particle-component pairs in the first region. The method selectively transfers at least one of the particle-component pairs to an isolated location in a second region where the particle-component pair receive an additional component of the material formed on the particle. The method thereby produces a first material associated with the first particle-component pair and a second material associated with the second particle-component pair.

### BRIEF DESCRIPTION OF THE DRAWINGS

FIG. 1 is a top view of a first region wherein all of the pockets of the region retain a particle.

FIG. 2 is a section view of the first region of FIG. 1 taken through one row of pockets.

FIG. 3 is a top view of a first mask used in transferring a selected portion of the particles retained in the pockets of a first region (FIG. 1) to the pockets of a second region (FIG. 4).

FIG. 4 is a top view of a second region wherein a portion of the pockets retain selected particles transferred from a first region (FIG. 1 or FIG. 5).

FIG. 5 is a top view of the first region after a selected portion of the particles retained in the pockets have been transferred to a second region.

FIG. 6 is a top view of a second mask used in transferring a selected portion of the particles retain retained in the pockets of a first region (FIG. 1 or FIG. 5) to the pockets of a third region (FIG. 7).

FIG. 7 is a top view of a third region wherein a selected portion of the pockets retain particles selectively transferred from a first region (FIG. 1 or FIG. 5).

FIG. 8 is a section view of a region where the pockets of the region have a vessel.

FIG. 9 is a perspective view of a portion of an extraction tool that may be used in conjunction with region and vessels of FIG. 8

### DETAILED DESCRIPTION OF THE INVENTION

The present invention provides an efficient method of assembling an array of different discrete particle materials where materials having known components are at spatially identifiable locations on regions. At least two regions are used in the method, and more than two regions may be employed. The regions retain the particle and at least a first component placed on or reacted with the particle. The regions contain spatially identifiable locations for assembly of individual materials. Spacing the locations apart and isolating them prevents contamination during by production of materials by delivery of selected components to the locations.

Essentially, any conceivable region having a rigid or semi-rigid surface can be employed in the invention. A region can comprise a multiplicity of different surfaces grouped into a region by a predetermined association. More typically a unitary substrate with multiple locations defined upon its surface will define a region or multiple regions. The region can be organic, inorganic, biological, nonbiological, or a combination of any of these, existing as particles, strands, precipitates, gels, sheets, tubing, spheres, containers, capillaries, pads, slices, films, plates, slides, etc. The region can have any convenient shape, such a disc, square, sphere, circle, etc. In many embodiments, at least one surface of the region will be substantially flat with physically separate isolated locations for retaining different particles and components using, for example, pockets in the form of dimples, wells, depressed portions, raised portions, etched trenches, or the like. In some embodiments, the region itself contains wells, raised regions, etched trenches, and such which form all or part of the isolated locations while in other embodiments another component may be used in combination with the region to form the isolated locations. The isolated locations are spatially identifiable and therefore it is preferred that the multiple regions have the same format or at least a portion of the same format.

The region may be any of a wide variety of materials including, for example, polymers, plastics, pyrex, quartz, resins, silicon, silica or silica-based materials, carbon, metals, inorganic glasses, inorganic crystals, membranes, etc. Other region materials will be readily apparent to those of skill in the art. Surfaces on the solid region that provide the location can be composed of the same materials as the region or, alternatively, base materials can be coated with a different material such as an adsorbent, for example, cellulose, to which the components of interest are delivered. The most appropriate region and region-surface materials will depend on the class of materials to be assembled.

The method, when used most effectively, employs small areas for location and large number of locations in the regions to assemble large numbers of different materials. In most embodiments, an isolated location on the region and, therefore, the area upon which each distinct particle material is assembled is smaller than about 5 cm², preferably less than 1 cm², more preferably less than 1 mm², and still more preferably less than 0.5 mm². In most preferred embodiments, the regions have increasing numbers of locations 10, 100, 1,000, and so on.

In preferred embodiments, each single region has at least 10 different particle materials and, more preferably, at least 100 different particle materials assembled thereon. In other embodiments, a single region may have more than 1000 particle materials assembled thereon. In some embodiments, the delivery process is repeated to provide particle materials with as few as two components, although the process may be readily adapted to form materials having 3, 4, 5, 6, 7, 8 or more components therein.

As previously explained, each region preferably has a surface with isolated locations for particle retention and material assembly, but the regions may take on a variety of alternative surface configurations. Regardless of the configuration of the region surface, it is important that the materials, once assembled in the individual isolated locations, be prevented from moving to adjacent isolated locations. Sufficient amount of space between the isolated locations on the region as well as restraints to inhibit particle movement operate to prevent or restrict interdiffusion of the various material between isolated locations as desired. A mechanical device or physical structure define the various isolated locations on the region. For example, arrangements that produce the pocket type structures such as a wall or other physical barrier can be used to inhibit the particles or components in an isolated location from moving to adjacent isolated location. Other suitable structures include, a sufficiently deep dimple or other recess can be used to prevent the components and particles in the individual isolated locations from moving to adjacent isolated locations. More elaborate arrangements may retain particles for selective release and transfer. Such systems could include vacuum orifices to pneumatically restrain particles on surfaces defining the orifices or electrostatic charges to appropriately insulated pockets to isolate the charge delivery as desired. Some embodiments of the invention deliver a same component to each isolated location of the region and in those embodiments interdiffusing of that component across multiple isolated locations is not of concern. However, other embodiments deliver different components to different isolated locations of a regions and in those embodiments, the delivered components as well as the assembled material are prevented from entering an isolated location other than the targeted isolated location.

At least one solid particle is retained in each isolated location of the first region. The particle allows for easy physical manipulation and transfer of a composite from one region to another. To facilitate handling, the particle will typically have a dimension of at least one dimension with a length of 100 microns or more. The particle may be inert to additional components or may be reactive with additional components. Preferably the particle can be a discrete amount of small beads or pellets or any solid particle having a diameter in a range of 100 microns to 10 mm and more preferably having a diameter of 0.5 to 3 mm. The number of particles used will depend on the number of materials being assembled and can range anywhere from 2 to an infinite number of particles. It is preferred to use a single particle for each material made, but isolated locations may retain multiple particles. Preferably where a location retains multiple particles, all of the particles from one location remain together as they are transferred to the next location. Preferably the particles at each location have sufficient size and integrity to permit ready removal of the entire particle or all of the particles so that the removal leaves no residue.

Example of suitable particle materials include organic or inorganic materials including those commonly found as catalyst supports such as molecular sieves, zeolites, inorganic oxides, clays, inorganic sulfides and inorganic nitrides. Examples of inorganic oxides may include alumina, silica, zirconia, magnesia, chromia or boria. The particles may also be a mixture of two or more materials or may contain binding material. The particles may be of any shape including known catalyst shapes and irregular shaped particles.

The array of materials is prepared by delivering material components and particles to isolated locations on a first region, sorting the resulting composites, transferring at least one composite to an isolated location on a second region, and delivering at least an additional material component to the composite on the second region. For instance the array of materials is assembled as follows. Particles A are delivered to a first and a second isolated location on a first region. Material component B is delivered to the first isolated location on the first region thereby forming composite A-B. Material component C is delivered to the second isolated location of the first region thereby forming composite A-C. Composite A-C is transferred to a third isolated location which is on a second region. An additional material component D is delivered to the third isolated location on the second region thereby forming composite A-C-D. The assembly method may be repeated, with additional components, to form a vast array of components at spatially identifiable isolated locations of two or more regions. The particles received by the second region may originate from more than one region. For example the second region above may receive at two locations two particles E that both received a material component F when on a third region. On the second region one particle E may also receive component D while the other particle E receives component G so that the second region contains a different locations the composites A-C-D, E-F-D and E-F-G.

The components can be sequentially or simultaneously delivered to the isolated locations of the regions using any of a number of different delivery techniques Each component can be delivered in either a uniform or gradient fashion to produce either a single stoichiometry or, alternatively, a large number of stoichiometries at the different isolated locations of the regions.

The solid particles may be delivered to the isolated locations using any technique suitable for solid particle delivery, such as manual placement of the particles into the isolated locations, automated solid dosing equipment that physically holds the particles for movement and sorting between the different regions of the arrays or imparting forces on selected pocket that transport the particles from pockets in one region to pockets in another region.

As mentioned previously, the isolated locations may communicate with fluid ports for selective application of the fluid flow for selective transfer of the particles contained thereat. For example selective fluid withdrawal from a port located near a particle can retain that particle when placed over another region into which unrestrained particles will drop at a desired location. Rather than employ individually piped ports to all or group of location in the array, it may use pockets as the locations and common perforated bottom to define a semi-closed end of the pockets. By tipping the delivering array and the receiving array with the inlet end of the pockets facing upward and toward each other the arrays can accept an angled shaped transfer block between their opposing faces. The transfer block will define cross passages for each similarly located pocket or each array. The selective application of discrete fluid stream at the perforated bottom of selected pockets can send or draw selected particle up from the bottom of the delivering pocket and down into the bottom of the receiving pocket.

Another transfer method may employ electrostatic charges. Location in the form of flat surfaces or pockets may define a conductive portion surrounded by a suitably insulated portion for selective application of an electrical charge to individual locations or groups of locations. Selective application of the charge to particle locations may repel or attract particles as necessary to transfer particles in any manner necessary for controlling the location of the particles and tracking their position to produce an array that contain the desired materials at known locations.

Delivery of the fluid components is preferably accomplished using a dispenser where the fluid components are added to the isolated locations in the form of droplets. Commercially available equipment such as pipetting and micropipetting apparatus can be adapted to dispense droplet volumes of components into the isolated locations. It is preferred that the pipetter be automated. Another technique employs a solution depositing apparatus that resembles devices commonly employed in the ink-jet printing field. Such inkjet dispensers include, for example, the pulse pressure type, the bubble jet type and the slit jet type. The operation of these systems are well known in the art and not explained in detail here. Such ink-jet printers can be used with minor modification by simply substituting a component containing solution or powder for the ink as described in literature. Ink-jet printers having single or multiple nozzles can be used to deliver single or multiple material components to a single isolated location on a region or to multiple isolated locations on a region. As improvements are made in field of ink-jet printers, such improvements can be used in the methods of the present invention.

The fluid components can also be delivered to the isolated locations of the region using an electrophoretic pump in which a thin capillary connects a reservoir of the component with the nozzle of the dispenser. At both ends of the capillary, electrodes are present to provide a potential difference. As is known in the art, the speed at which a chemical species travels in a potential gradient of an electrophoretic medium is governed by a variety of physical properties, including the charge density, size, and shape of the species being transported, as well as the physical and chemical properties of the transport medium itself. Under the proper conditions of potential gradient, capillary dimensions, and transport medium rheology, a hydrodynamic flow will be set up within the capillary. Thus, bulk fluid containing the component to be delivered can be pumped from a reservoir to the region. By adjusting the appropriate position of the region with respect to the electrophoretic pump nozzle, the component solution can be precisely delivered to isolated locations on the regions.

It is also envisioned that a region may be immersed in a solution containing at least one component so that the isolated locations retain a portion of the solution containing at least one component.

The solid particle may be delivered to the isolated locations of the first region first, followed by the delivery of at least a first components. Alternatively, at least the first component may be delivered to each isolated location of a region in a first step, followed by the delivery of the particle to each isolated location of the region. As discussed above, components can be delivered to isolated locations on the region either sequentially or simultaneously, and the components can be simultaneously delivered to either a single isolated location on the region or, alternatively, to multiple isolated locations on the region. For example, using an ink-jet dispenser having two nozzles, two different components can be simultaneously delivered to a single isolated location on the region. Alternatively, using this same ink-jet dispenser, a component can be simultaneously delivered to two different isolated locations on the region. The same component or two different components can be delivered. The same component can be delivered to different isolated locations at either the same or different concentrations. Likewise, using an ink-jet dispenser having twenty nozzles, twenty different components can be simultaneously delivered to a single isolated location on a region or, alternatively, twenty identical or different components can be simultaneously delivered to twenty different isolated locations on a region.

In a classic split-pool format, all the particle-component pairs from the first region, if different, would likely be pooled together and then split into portions. Each portion would receive additional treatment that could include the incorporation of additional components. After each portion received additional treatment, the portions would be pooled again and then divided into portions yet again. The process would continue until a sufficient number of materials were formed. The materials would be evaluated and the identification of the materials of interest would require analysis or tagging during the formation.

In the present invention, the particle and component materials are assembled in a stepwise technique similar to classic split-pool, but without pooling the intermediates. Instead, at least one of the particle-component pairs assembled on the first region is transferred to a second region where an additional component is delivered. Often, a portion of the total number of particle-component pairs assembled on the first region are transferred to a second region where one or more additional components are added. As with the assembly on the first region, the same component in the same or different concentrations can be added to each isolated location on the second region, or different components can be added to each isolated location. Suitable delivery methods are as described above. One or more additional components can also be added to the particle-component pairs retained by the first region after the transferring step.

Masks can be used to aid in the transferring of the particle-component pairs from one region to another. For example, where the regions are well plates, a mask having perforations at corresponding locations to those particle-component pairs to be transferred from one region to another can be placed over the first region. The second region may be inverted and placed over the mask. The entire assembly can then be inverted so that the selected particle-component pairs fall from the first region, through the openings of the mask, and are retained in the second region. The mask and the first region are removed from the second region, inverted together and the mask removed. Those non-selected particle-component pairs remain retained in the first region. Other selective transfer techniques are readily apparent to those of ordinary skill in the art, ranging from the basic manual selection and manual physical transferring of particle-component pair by particle-component pair, to a more complex robotic or automated procedure transferring multiple particle-component pairs simultaneously.

The transferring can be repeated one or more times. Multiple portions of the particle-component pairs can be transferred in a sequential process, i.e,. with a first region having 100 isolated locations and retaining 100 particle-component pairs, a grouping of 25 of particle-component pairs can be transferred from the first region to a second region, another grouping of 25 of particle-component pairs can be transferred from the first region to a third region, yet another grouping of 25 of particle-component pairs can be transferred from the first region to a fourth region. A grouping of 25 of particle-component pairs can be retained on the first region. The result thus far is four regions, each region having a grouping of 25 of particle-component pairs. Various different steps can be employed at this point. For example, an additional component can be added to each of the four regions; other groupings from other regions may also be transferred to one or more of the four regions and retained in additional isolated locations; additional transfer steps may be performed after additional component(s) have been delivered, and so on.

One embodiment of the invention employs one or more algorithms to assist in sorting the particle-component pairs. Preferred algorithms maximize the number of different particle-component combinations in the minimum number of iterative steps.

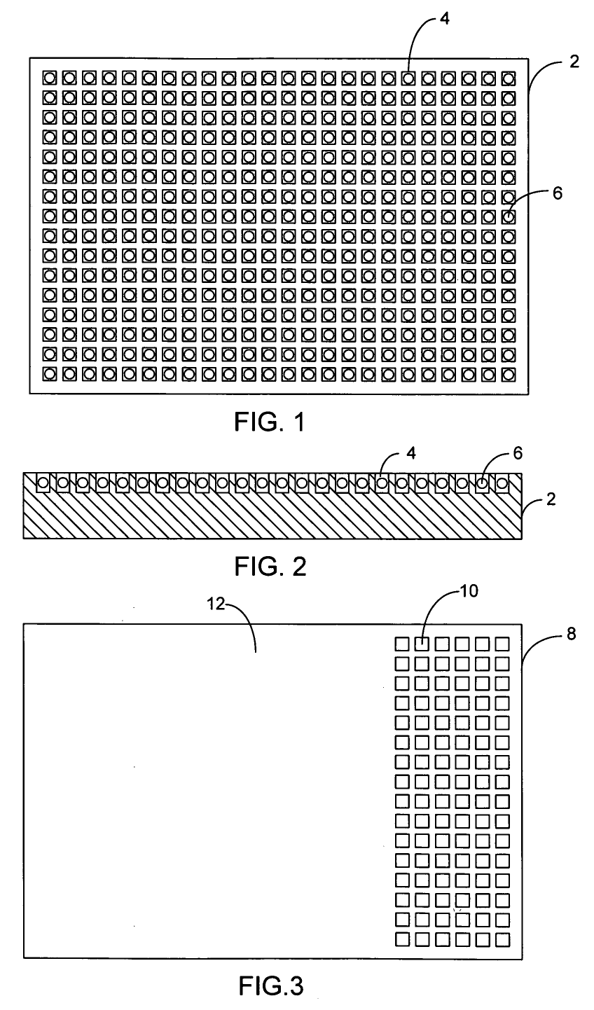

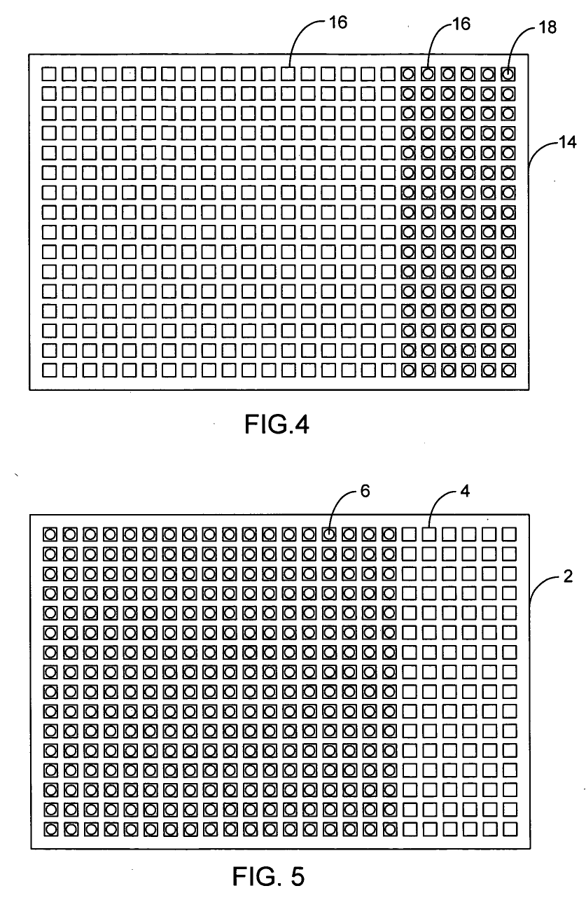

FIGS. 1-7 demonstrate an embodiment of transferring selected particle component pairs from pockets of a first region to pockets of a second and third region. FIG. 1 shows a top view of a first region 2 having 384 pockets 6 with each pocket retaining a particle 6 that may itself comprise a component and thus provide a particle-component pair or when combined with a component becomes a particle-component pair. FIG. 2 shows a sectional view of the first region 2 taken through one row of pockets 4. FIG. 3 shows a first mask 8 having through-going perforations 10 corresponding to the pockets 4 of the first region 2 retaining selected particle component pairs. Mask 8 has solid region 12 corresponding to the pockets 4 of the first region 2 for retaining non-selected particle component pairs. Mask 8 is placed over region 2 so that perforations 10 of mask 8 are aligned with selected pocket 4 of region 2. A second region having pockets corresponding to the pockets of selected particle component pairs in region 2 is inverted and placed over the mask 8. The second region is oriented so that the pockets of the second region are in position to accept transfer of the selected particle component pairs from the first region 2. Maintaining the alignment of the first region 2, the mask 8, and the second region, the assembly is inverted so that the selected particle-component pairs fall from the pockets 4 of the first region 2 through the perforations 10 of mask 8, and into corresponding pockets 16 of the second region 14 as shown in FIG. 4. Mask 8 and the first region 2 together are inverted again and mask 8 is removed from first region 2. FIG. 5 shows first region 2 having retained non-selected particle-component pairs in pockets 4 since transfer of non-selected particle component pairs were blocked by solid region 12 of mask 8.

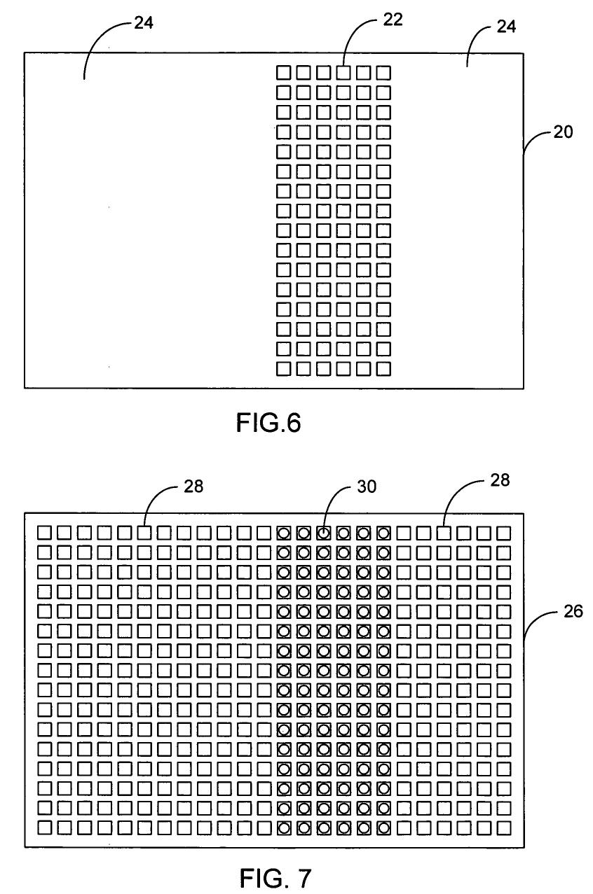

A second transfer step involves placing mask 20, shown in FIG. 6, over the first region 2 shown in FIG. 5. Through going perforations 22 of mask 20 correspond with particle component pairs retained in first region 2 that are selected for transfer, while solid portions 24 of mask 20 correspond with particle component pairs retained in first region 2 that are not selected for transfer. Mask 20 is placed over first region 2 so that perforations 22 of mask 20 are aligned with selected pockets 4 of region 2. A third region having pockets corresponding to the pockets of selected particle component pairs in region 2 is inverted and placed over the mask 20. The third region is oriented so that the pockets of the third region are in position to accept transfer of the selected particle component pairs from the first region 2. Maintaining the alignment of the first region 2, the mask 20, and the third region, the assembly is inverted so that the selected particle-component pairs fall from the pockets 4 of the first region 2 through the perforations 22 of mask 20, and into corresponding pockets 28 of the third region 26 as shown in FIG. 7. Mask 20 and the first region 2 together are inverted again and mask 20 is removed from first region 2. First region 2 will have retained non-selected particle-component pairs in pockets since the transfer of non-selected particle component pairs were blocked by solid region 24 of mask 20. Repeated transfers may be conducted in a similar manner. Additional component may be added to the particles retained in the first region or transferred to the second or third regions.

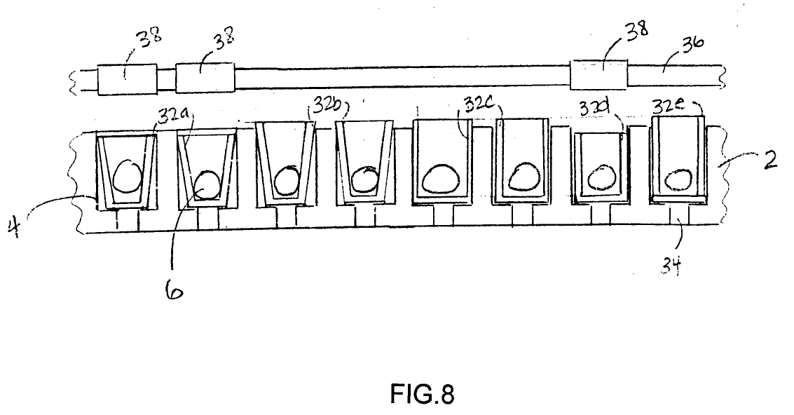

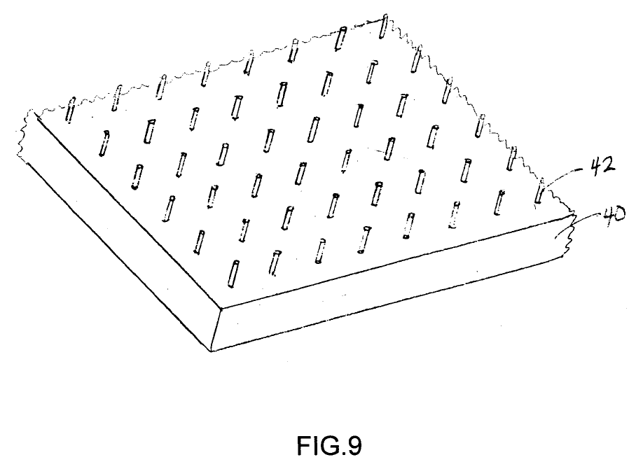

FIGS. 8 and 9 show an embodiment of the invention that employs an optional vessel at one, two, or more, or all of the pockets of at least one regions. The vessels may operate in connection with an extraction tool such as that shown in FIG. 9 in order to simultaneously disengage all vessels from the pockets. The optional vessels 32 as shown in FIG. 8 (which is enlarged to show detail as compared to FIGS. 1-7) provide several advantages with the most important being the simple means of removing the vessel from the pocket. This allows for a greater degree of flexibility in that different vessels may be grouped for different types of experiments. Another benefit is the ease of extracting the products from the separate vessels as compared to extracting multiple solid products from a unitary device. Yet another advantage is the significantly reduced chance of cross contamination between runs using the multiple vessels. Small vessels may improve operations by eliminating the need to clean small confined pockets in regions and thereby eliminating the risk of undetected contamination compromising future experiments. The individual vessel may also be used to weigh the reagents and or products to a high degree of accuracy. The vessels may also provide an alternative approach to product recovery through using channels 34 in the region 2. Vessels 32 containing synthesis products may be pressed out of the pockets 4 of the region 2 using an extraction device which is discussed in greater detail below.

FIG. 8 also shows optional retaining plate 36 that contains optional lids 38 to urge the bottom of vessels 32 into contact with the bottom of the respective pockets 4 in region 2. Retaining plate 36 may also operated to urge a portion of lid 38 into the interior portion of vessel 32. The lids may actually be an integral part of the retaining plate, or the retaining plate may retain a separate lid for each vessel that has a lid. The vessels 32 are removably placed within pockets 4 defined by region 2. The vessels may be removably placed about the region before, during, or after the components have been introduced. The vessels may be removably placed about the region sequentially, at the same time, or in groups. By removably placed about the region, it is meant that the vessels are placed within, on, or against the region. In one embodiment, a pocket contains no more than one vessel. The term about a region is meant to include within, on, or against the region.

A variety of vessel configurations and functional interaction between the vessel and the region are possible, some of which can restrain the movement of the vessels with respect to the region. FIG. 8 shows a variety of vessels occupying the pockets and a retaining plate with regions adapted to provide a suitable lid for use with the particular vessel configuration covered by that region. In FIG. 8, region 2 supports a variety of different vessels 32. At the location of each vessel region 2 further defines channels 34. Retaining plate 36 retains lids 38. Vessels 32a have tapered geometries where a closed end has a circumference less than that of an open end and vessels 32a are contained within the pockets 4 of region 2 with the exterior surface of the vessel about its open end contacting the surface of the pocket.

Vessels 32b also have tapered geometries where a closed end has a circumference less than that of an open end. Vessels 32b, while positioned within the pocket, extend beyond the opening of the pocket in the region. The exterior surface of vessels 32b are in contact with the pocket opening. The tapered vessels do not need retaining plate 36 or lids 38 as a restraint against rotation or movement. Although not required, the tapered vessels may be force-fit within the pockets and frictional forces operate against rotation or other movement such as translational movement. However, the retaining plate and or lids may be used to prevent cross contamination or to contain materials within the vessel during mixing.

Vessels 32c have cylindrical or rectangular geometries and while positioned within the pocket, extend beyond the opening of the pocket in the region. Vessel 32d has a cylindrical or rectangular geometry and is contained completely within the pocket of region 2 such that lid 38 is partially inserted within the pocket before contacting vessel 32d. Vessel 32e, also having a cylindrical or rectangular geometry, is a two-piece vessel comprised of a bottom plate or disk in combination with sides or a sleeve. As with the unitary vessels, vessel 32e may be contained within the pocket, or may extend beyond the pocket as shown. Although not required, any of the vessels may be force-fit within the pockets as described above for the tapered vessels in order to restrict against rotation or other movement.

Force-fit vessels, or any vessels that become unexpectedly lodged within a pocket may be extracted from the region using an extraction tool such as that shown in FIG. 9. With reference to FIG. 9, extraction tool 44 has jig 40 that positions pins 42 for alignment with channels 34 of the pockets in order to disengage the vessels from the pockets. The extraction tool provides for simultaneous disengagement of the vessels from the pockets. In one embodiment of the invention, to extract the vessels, the region containing the vessels within the pockets would be placed over the extraction tool and forced downward so that pins 42 enter channels 34 and contact vessels 32. Continued force would result in disengagement of the vessels 32 from the pockets of the region 2. Alternate means of removing the vessels from the pockets are within the scope of the invention. For example, the vessels could be manually extracted or mechanical or vacuum tools could be employed.

### EXAMPLE

An experiment demonstrating the steps of the invention was conducted using food coloring and water solutions and alumina beads. The regions were prepared by manually placing a single bead in each of the 384 recessed locations of each of four regions, region 1, region 2, region 3 and region 4. The recessed locations formed a grid pattern of 24 rows and 16 columns in one surface of the region. Four baths were prepared, three of which containing approximately 10 drops of commercially available food coloring, yellow, red, and blue, in approximately 100 ml of water and a fourth bath containing only approximately 100 ml of water. The solutions in the bath were deep enough so that the regions could be submersed and each bead retained in the regions would contact the solution.

Each region was placed within one of the baths and the beads retained in the region were allowed to contact the solution in the bath for 30 minutes. The regions were removed from the baths and the beads allowed to air dry for 30 minutes and then oven dry at 60° C. for 1 hour. Region 1 was contacted with the bath containing only water and therefore retained 384 beads that remained their original white color. Region 2 was contacted with the bath containing yellow food coloring and water and therefore retained 384 beads that were yellow in color. Region 3 was contacted with the bath containing water and red food coloring and therefore retained 384 beads that were red in color. Region 4 was contacted with the bath containing water and blue food coloring and therefore retained 384 beads that were blue in color.

The beads retained by Region 1 were divided into four portions using row sorting where each portion containing the beads in all 16 columns of 6 rows. Each portion was transferred to corresponding isolated locations of an additional four regions, Region 1′, Region 2′, Region 3′, and Region 4′. Similarly, the beads retained on Regions 2, 3, and 4, were each divided into four portions with each portion containing the beads in the 16 columns of 6 rows of each respective region. The portions of beads from Regions 2, 3, and 4 were transferred to Regions 2′, 3′, and 4′. The resulting Regions 1′, 2′, 3′, and 4′ each retain a 6×16 (row by column) portion of white beads, a 6×16 portion of yellow beads, a 6×16 portion of red beads and a 6×16 portion of blue beads.

Region 1′ was submersed in the bath containing only water, and the beads retained in Region 1′ were allowed to contact the solution in the bath for for 30 minutes. Region 1′ was removed from the bath and the beads allowed to air dry for 30 minutes and then oven dry at 60° C. for 1 hour. Region 1′ after contacting the bath retained the same 96 white beads, 96 yellow beads, 96 red beads, and 96 blue beads since the bath used to contact Region 1′ had no food coloring.

Region 2′ was submersed in the bath containing yellow food coloring in water, and the beads retained in Region 2′ were allowed to contact the solution in the bath for 30 minutes. Region 2′ was removed from the bath and the beads allowed to air dry for 30 minutes and then oven dry at 60° C. for 1 hour. Region 2′ after contacting the bath retained 96 yellow beads (originally white), 96 deeper yellow colored beads, 96 orange (yellow-red) beads, and 96 green (yellow-blue) beads since the bath used to contact Region 2′ had yellow food coloring.

Region 3′ was submersed in the bath containing red food coloring in water, and the beads retained in Region 3′ were allowed to contact the solution in the bath for 30 minutes. Region 3′ was removed from the bath and the beads allowed to air dry for 30 minutes and then oven dry at 60° C. for 1 hour. Region 3′ after contacting the bath retained 96 red beads (originally white), 96 deeper red colored beads, 96 orange (red-yellow) beads, and 96 purple (red-blue) beads since the bath used to contact Region 3′ had red food coloring.

Finally, Region 4′ was submersed in the bath containing blue food coloring in water, and the beads retained in Region 4′ were allowed to contact the solution in the bath for 30 minutes. Region 4′ was removed from the bath and the beads allowed to air dry for 30 minutes and then oven dry at 60° C. for 1 hour. Region 4′ after contacting the bath retained 96 blue beads (originally white), 96 deeper blue colored beads, 96 purple (blue-red) beads, and 96 green (blue-yellow) beads since the bath used to contact Region 4′ had blue food coloring.

Note that each time a region was submersed in a bath while the solution contacted each isolated location, each bead was retained by the region and prevented from contacting another bead retained by the region.

The process was repeated using another four regions, Region 1″, 2″, 3″and 4″. In this iteration however, the beads retained by Regions 1′, 2′, 3′ and 4′ were divided into four portions using column sorting where each portion containing the beads in all 24 rows of a selected 4 columns. Each portion was transferred to corresponding isolated locations of the additional four Regions, 1″, 2″, 3″and 4″. Again, each region was submersed in the water and water-food coloring baths as described above. Additional combinations of bead-ring were formed on each region.

What is claimed is:

1. A method of assembling an array of materials with each different material identified by the location of at least one discrete particle:
retaining a first particle component at a first spatially identifiable location in a first region;
retaining a second particle component at a second spatially identifiable location in the first region with the second spatially identifiable location spaced apart and isolated from the first spatially identifiable location;
delivering a first selected component to the first spatially identifiable location;
delivering a second selected component to the second spatially identifiable location;
selectively transferring at least one of the first particle-component pair or the second particle-component pair to a third spatially identifiable location in a second region and retaining the thus transferred particle-component pair in the third spatially identifiable location;
delivering an additional component to the third spatially identifiable location; and producing a first material associated with the first particle component and a second material associated with the second particle component.

2. The method of claim 1 wherein the first selected component is delivered to the first spatially identifiable location before delivery of the first particle component for retention at the first spatially identifiable region.

3. The method of claim 1 wherein the particles have at least one dimension of 100 micron or more.

4. The method of claim 1 wherein at least one of the first selected component or the second selected component reacts with the first particle component to produce the first material.

5. The method of claim 1 wherein the number of particle components at a spatially identifiable location does not exceed 100.

6. The method of claim 1 wherein each region has at least 10 spatially identifiable locations.

7. The method of claim 1 wherein at least two components in at least two spatially identifiable locations of at least one of the regions undergo simultaneous reaction.

8. The method of claim 1 wherein the first selected component, the second selected component and the additional component all differ in at least one of composition or concentration.

9. The method of claim 1 wherein a third particle component is retained in a third spatially identifiable location in a third region and a third selected component is delivered to the third spatially identifiable location to produce a third particle-component pair and the third particle-component pair is transferred to a fourth spatially identifiable location in the second region and a further component is delivered to the fourth spatially identifiable region to produce a third material associated with the third particle component.

10. The method of claim 1 wherein a pocket at least partially defines each spatially identifiable location and each pocket can at least partially retain a particle component at the a spatially identifiable location.

11. The method of claim 1 wherein the first region and the second region are located on different substrates.

12. The method of claim 11 wherein the particle components are retained by individual pockets on the different substrates and particle components are selectively transferred by retaining selected particle components on one substrate while allowing the transferred particle components to move into selected pockets of another substrate.

13. The method of claim 12 wherein a transfer mask is used to occlude the openings of the pockets that retain non-transferred particle components and provide transfer paths for particle components to drop from the pockets that transfer particle components to the pockets that receive the transferred particle components.

14. The method of claim 12 wherein fluid ports communicates with the pockets of the substrate that selectively retains particle components and the selected particle components are pneumatically retained in or transferred from the desired pockets by fluid flow through the port.

15. The method of claim 12 wherein an isolated electrostatic charge is selectively imposed on individual pockets or groups of pockets to selectively retain particles in the selected pockets or selectively transfer particles to selected pockets.

16. The method of claim 1 wherein at least one of the regions retains at least one vessel at one of the spatially identifiable locations and the vessel is adapted to receive at least one discrete particle.

17. A method of making an array of materials with different material on discrete particles:
delivering a first component of a first material to a first pocket on a first substrate for contact with a first particle located therein;
delivering a first component of a second material to a second pocket on the first substrate for contact with a second particle located therein;
transferring the first particle to a first pocket on a second substrate and delivering a second component of the first material to the first pocket on the second substrate for contact with the first particle to produce a first material on the first particle; and,
producing a second material on the second particle by transferring the second particle to a second pocket on the second substrate and delivering a second component of the second material to the second pocket on the second substrate for contact with the second particle or by transferring the second particle to a first pocket on a third substrate and delivering a second component of the second material to the first pocket of the third substrate for contact with the second particle.

18. The method of claim 17 wherein the particles have a diameter in the range of 100 microns to 10 mm.

19. The method of claim 17 wherein each substrate retains particles at 8 or more locations

20. The method of claim 17 wherein components in different pockets on the same substrate undergo simultaneous reaction with particles or other components in the same pocket on that substrate.

21. The method of claim 17 wherein the first component of the first material differs from the first component of the second material and/or the second component of the first material differs from the second component of the second material.

22. A method of making an array of materials with different material on discrete particles:
delivering at least one component member in a series component members for producing a plurality of materials to a different pocket in a plurality of pockets defined by a first substrate for each component member to contact with a particle located therein;
transferring a portion of the particles from the pockets of the first substrate to a plurality of pockets in a second substrate and tracking the new location of each transferred particle in the second substrate;
delivering at least one additional component member in the series of component members to the pockets of the second substrate; and,
recovering a plurality of different materials with each different material incorporating a particle from a different pocket and the properties of each different material identified at least in part by the location of the pocket to which the particle was delivered.
